(19) United States
(12) Patent Application Publication
(10) Pub. No.: US 2009/0182582 A1
(43) Pub. Date: Jul. 16, 2009

(54) SYSTEM AND PORTABLE APPARATUS FOR SECURELY DISPENSING AND MAINTAINING ACCURATE INVENTORY OF CONTROLLED SUBSTANCES IN A HOSPITAL SETTING

(75) Inventor: Evelyn Hammon, Humansville, MO (US)

Correspondence Address: Workman Nydegger;1000 Eagle Gate Tower, 60 East South Temple, Salt Lake City, UT, 84111, US

(73) Assignee: HAWKEYE PRODUCTION LLC, Humansville, MO (US)

(21) Appl. No.: 12/354,367

(22) Filed: Jan. 15, 2009

Related U.S. Application Data
(60) Provisional application No. 61/021,096, filed on Jan. 15, 2008.

(51) Int. Cl. G06Q 50/00

(52) U.S. Cl. 705/3; 705/2

(57) ABSTRACT

A system for use in a hospital or other medical facility that protects medications (e.g., injected or orally administered) against diversion by facility personnel when transported from a central pharmacy to the patient. The system includes a portable hand-held carrier to encapsulate the medication, which may be contained within a vial, cartridge, or other container. The hand-held carrier protects and prevents removal or administration of the medication until after it has been transported to the patient. Wireless RFID or similar technology unlocks the hand-held carrier and enables injection, dispensing or other administration at the patient's bedside. Electronics in a bedside portion of the system communicate through a network to log the injection or other administration event in a central database.

### RELATED APPLICATIONS

The present application claims the benefit of U.S. Provisional Patent Application Ser. No. 61/021,096, filed Jan. 15, 2008, and entitled “SYSTEM AND PORTABLE APPARATUS FOR SECURELY DISPENSING AND MAINTAINING ACCURATE INVENTORY OF CONTROLLED SUBSTANCES IN A HOSPITAL SETTING”, the disclosure of which is incorporated in its entirety.

### BACKGROUND OF THE INVENTION

1. The Field of the Invention

The present invention is directed to the field of medication dispensing. More particularly, the present invention is directed to a system for securely dispensing and maintaining an accurate inventory of controlled substances in a hospital or similar setting.

2. The Relevant Technology

The medical service community stores, transports, and administers controlled substances (e.g., injection administered medications as well as orally administered medications) in response to doctor-ordered treatment authorized via prescriptions. Managing this operation currently requires periodic inventory of all controlled substances so as to balance the inventory as a result of drug administration. As currently conducted, this operation creates opportunities for personnel authorized (e.g., registered nurses and other medical providers) to handle a controlled substance to divert it in an unauthorized manner to either their own illegal use or for illegal resale.

Although the extent of drug and alcohol abuse within the nursing profession has not been fully documented, at least one industry journal has indicated that as much as 10 percent of the nursing population has an issue with drug and/or alcohol abuse, and that 6 percent of this population has a sufficiently serious problem that it interferes with their ability to safely practice in the field (see “Substance Abuse Among Nurses—Defining the Issue”, AORN Journal, October 2005, Debra Dunn).

In addition, current methods of inventory and control rely heavily on human input, and as such are prone to medical errors. For example, in a small but significant fraction of cases, either the wrong medication or incorrect dosage is administered to a patient, which can result in serious harm or even death to the patient.

As such, it would be advantageous to provide a comprehensive system for maintaining control and accurate inventory over controlled substances used within a hospital or similar setting. It would be an added advantage if the system would help prevent medication errors in addition to preventing theft or illegal diversion of controlled substances.

### SUMMARY OF THE PREFERRED EMBODIMENTS

The present invention is directed to a system for use in a hospital or other medical facility. The system protects medications (e.g., injected or orally administered) against unauthorized diversion by facility personnel when transported from a central pharmacy to the patient. The system includes a portable hand-held carrier that encapsulates the medication, which may be contained within a vial, cartridge, or other container (e.g., a needle-mounted vial system such as Carpuject® from Hospira/Abbott Labs, or Tubex™ from Wyeth Ayerst Labs). The hand-held carrier protects and prevents removal or administration of the medication until after it has been transported to the designated patient. Wireless RFID or similar technology unlocks the hand-held carrier and enables injection, dispensing or other administration at the patient's designated location (e.g., bedside). Electronics in the designated location portion of the system communicate through a network to log the injection or other administration event into a central database. The device may advantageously provide visual and voice-response feedback confirming the contents of the vial, both in drug type and dosage.

The system of the present invention advantageously reduces or eliminates drug theft in hospitals, reduces or eliminates medication errors of drug type and/or dosage, and can be accomplished in a cost-effective manner so as to be readily adopted by hospitals and other medical facilities of moderate or even relatively small size.

These and other benefits, advantages and features of the present invention will become more full apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1A is a perspective view of an exemplary central system depository for controlled medications including an exemplary hand-held portable carrier for securely transporting medication from the central depository to a patient;

FIG. 1B is a close up perspective view better illustrating the exemplary hand-held portable carrier of FIG. 1A;

FIG. 2 is a perspective view of the exemplary hand-held carrier of FIGS. 1A-1B and a charging and programming receptacle configured to receive a proximal end of the hand-held carrier;

FIG. 3 is a close up perspective view of the exemplary hand-held carrier of FIG. 2;

FIG. 4 illustrates the distal end of the exemplary hand-held carrier in an unlocked and open position so as to allow loading of a vial, cartridge, or other medication container;

FIG. 5 is a top view of the exemplary hand-held carrier of FIG. 2;

FIG. 6 is a longitudinal cross-sectional view of the exemplary hand-held-carrier of FIG. 2;

FIG. 7 is a side view of the exemplary hand-held carrier of FIG. 2;

FIG. 8 is a bottom view of the exemplary hand-held carrier of FIG. 2;

FIG. 9 is a distal front end view of the exemplary hand-held carrier of FIG. 2;

FIG. 10 is a proximal back end view of the exemplary hand-held carrier of FIG. 2;

FIG. 11 is a cross-sectional view of the exemplary hand-held carrier of FIG. 2 in which the vial or other container is fully loaded; and

FIG. 12 is a cross-sectional view of the exemplary hand-held container of FIG. 2 in which the vial or other container has been fully emptied.

### DETAILED DESCRIPTION OF THE PREFERRED EMBODIMENTS

### I. Introduction

The system of the present invention according to one exemplary embodiment includes a central depository station for storing a plurality of medications. The central depository includes a computer for providing access to a central depository management database, which computer is connected through a network to terminals within a plurality of patient rooms, and one or more hand-held carrier devices. Each hand-held carrier device is configured to securely hold medication (e.g., stored within a vial, cartridge, or other container loaded into the device at the central depository station) for transport from the central depository to patient locales. For example, a typical installation in a large hospital including about 500 beds might be divided into several departments having about 20 to about 30 beds each. Each department might include 1 central depository station and one room terminal for each bed. Of course, if there were multiple beds in a single room, it may be possible to share a room terminal with multiple (e.g., 2) beds. In other words, such a hospital may include about 500 patient room terminals and about 50 carrier devices (e.g., 2 to 3 carrier devices per central depository station).

### II. Overview of Exemplary System for Maintaining Accurate Inventory

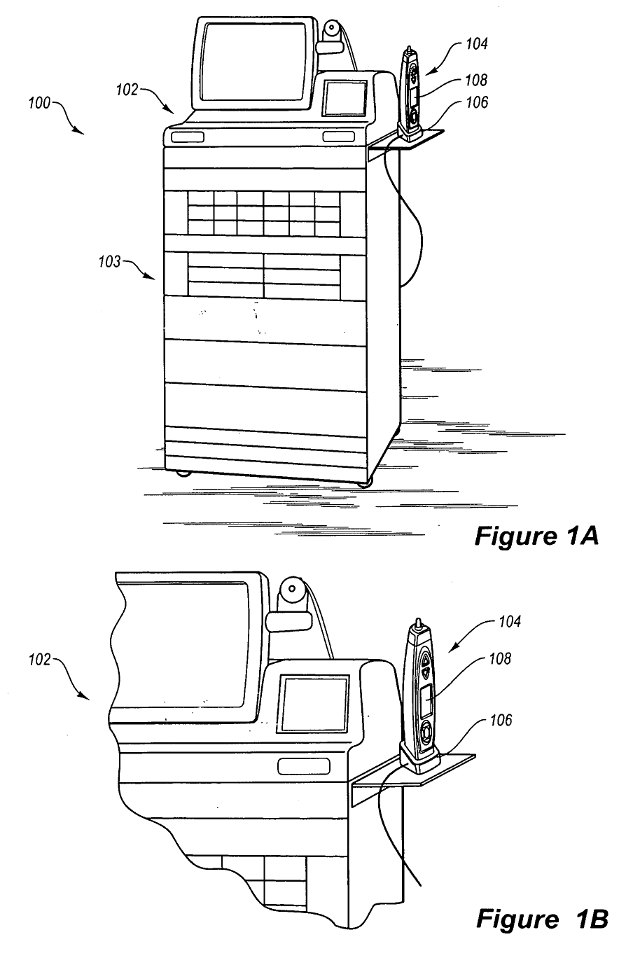

FIGS. 1A and 1B illustrate an exemplary central depository 100 including a computer 102 that includes a database that maintains an instant inventory of all medications allocated to it. Such a database maintaining an instant and continuously accurate inventory is advantageous as it is not required to manually inventory the controlled substances every time there is a nurse shift change (e.g., every 12 hours). The central depository station 100 may be located in the hospital pharmacy. In response to doctor orders, personnel in the pharmacy connects an empty hand-held carrier device 104 to a data port interface 106 (which may also charge a battery power source within carrier device 104) of the network at the central depository 100, removes the appropriate medication from inventory, and places it in the carrier 104. During this process, a bar code or other identification on the vial or other container of medication may be scanned or otherwise entered into the database system. Information relating to the medication type and dosage is saved to the central database as well as within the memory of the hand-held carrier 104. The hand-held carrier 104 may advantageously include a display 108 so as to show medication type and dosage. Additional information (e.g., patient name, room number) may also be viewed within the electronic display (e.g., by a scrolling feature). Preferably, the system includes multiple data port interfaces 106 for connecting multiple carriers 104.

Pharmacy personnel then closes the carrier 104 to lock it and disconnects the hand-held carrier from the terminal port 106. The central computer logs the various events as they occur, e.g., connection of empty carrier 104 to central depository port terminal 106, scan of medication, medication loaded, lock and disconnect of the hand-held carrier 104.

The nurse transports the carrier to the patient room, holds the carrier near the room terminal, and pushes a button on the carrier (e.g., in an example where the room terminal includes the ability to communicate wirelessly, for example, by RFID with the carrier). Other communication mechanisms may alternatively be employed (e.g., wired connection). If the medication is in the designated location and/or in close proximity to the designated patient, the carrier 104 unlocks and allows the nurse to proceed with the injection or administration of the controlled substance.

Through the network connection (e.g., wireless RFID), the central computer 102 logs these events and confirms that the proper medication has been delivered to the designated patient as ordered. If the carrier 104 is not in the authorized location, the carrier 104 may remain locked and/or provide a visual and/or auditory alarm. If the carrier 104 is unlocked in the designated location, it may then provide a voice announcement stating the medication type and dosage. This information could alternatively or additionally be provided visually (e.g., on display 108). After the injection, the nurse again holds the carrier 104 near the room terminal and pushes a button. This logs the event that the nurse indicates the injection or other medication administration has been completed.

Significant passage of time between unlock and completion of the injection or other administration event may indicate possible attempts at drug diversion. In one embodiment, if the nurse delays too long (e.g., more than about 6-7 minutes) the carrier may relock and/or provide an alarm (e.g., visual, auditory, and/or inform a supervisor that unauthorized diversion may be occurring). In another embodiment, such an alarm may be provided if the carrier is taken too far away from the central depository station and/or patient room (e.g., more than 500 feet from the central depository station and/or patient room). After the administration has been completed, the nurse returns the carrier to the central depository in the pharmacy for sanitizing, after which the carrier 104 is returned to inventory so that it may be reused. The central computer 102 logs this event to finalize the process. The central database 102 is instantly accurate at all times and has the capability of indicating how much medication is in-route at any given time. This is particularly advantageous as it is not required to manually inventory the controlled substances, and taking an inventory at the end of one nurse shift and the beginning of another can be quickly accomplished (e.g., a matter of seconds to a minute or two as compared to 30 minutes or more for a typical inventory performed at the overlap of each nurse shift change).

The hand-held carrier 104 may advantageously contain a barcode or other ID scanner which serves to confirm that the identification band of the patient matches the medication order. Optionally, the carrier 104 may provide an audible and/or visual response and/or additional information (e.g., type and dosage of the loaded medication).

### III. Exemplary Hand-Held Carrier Device

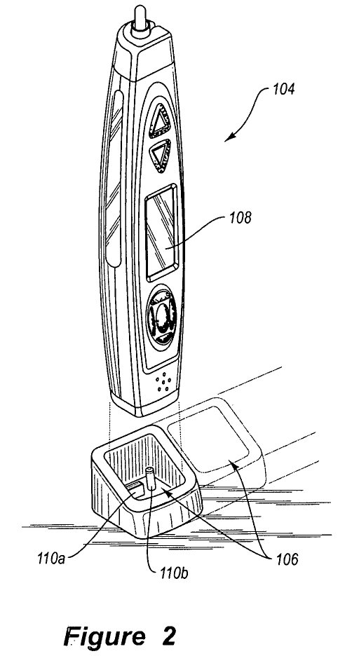

FIG. 2 illustrates an exemplary hand-held carrier 104 adjacent to a data port interface 106. As illustrated, a plurality of data port interfaces 106 may be included at central depository station 100. An exemplary interface 106 may include a data connector 110a (e.g., connected to a USB, serial, or other data interface of computer 102) and a power connector 110b for charging a power source (e.g., a battery) within carrier 104 when carrier 104 is mounted within data port interface 106.

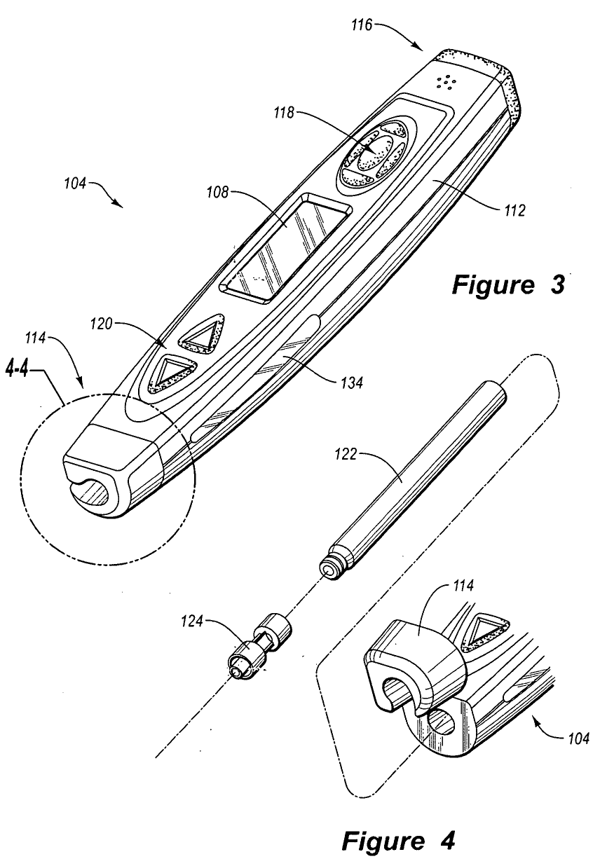

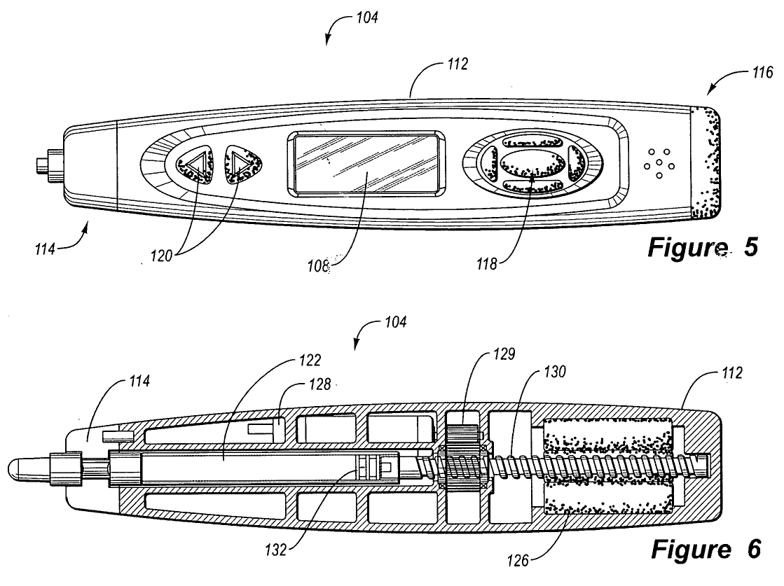

FIGS. 3-12 illustrate various views of carrier device 104. The hand-held carrier device as illustrated is advantageously small, battery-powered dispensing device that monitors and drives the dispensing (e.g., administration by hypodermic injection) under nurse control. The illustrated example of carrier device 104 includes an elongate body 112 extending between a distal delivery end 114 and a proximal gripping end 116. An electronic display 108 may be provided to display information relative to the contents of the carrier, the patient, etc. A plurality of buttons for providing input are also provided. For example, buttons 118 may be used for scrolling through information on display 108, and/or making a selection, while buttons 120 may be used to advance or retract a plunger or other mechanism for delivering medication held within carrier 104.

FIG. 4 illustrates distal end 114 rotated to an open position where it may receive a vial, cartridge or other container 122 to be loaded into carrier 104. Where the controlled substance within container 122 is to be injected, a connector (e.g., a luer connector) 124 may be coupled to the distal end of container 122. As seen in FIG. 6, body 112 may be formed of a rubber like grippable material. Various components of the system are provided within carrier 104. For example, a battery 126 is provided for powering display 108 as well as a motor 128 coupled to reduction gears 129, which are in turn coupled to a threaded plunger shaft 130. A distal end of plunger shaft 130 includes a plunger 132 configured so as to force a controlled substance within container 122 out distal end 114 (e.g., through a needle 138—illustrated as capped in FIG. 6). Although illustrated with specific components (e.g., battery 126, motor 128, gears 129, shaft 130, plunger 132) in specific locations and arrangements, it will be understood that other components and/or arrangements may be employed.

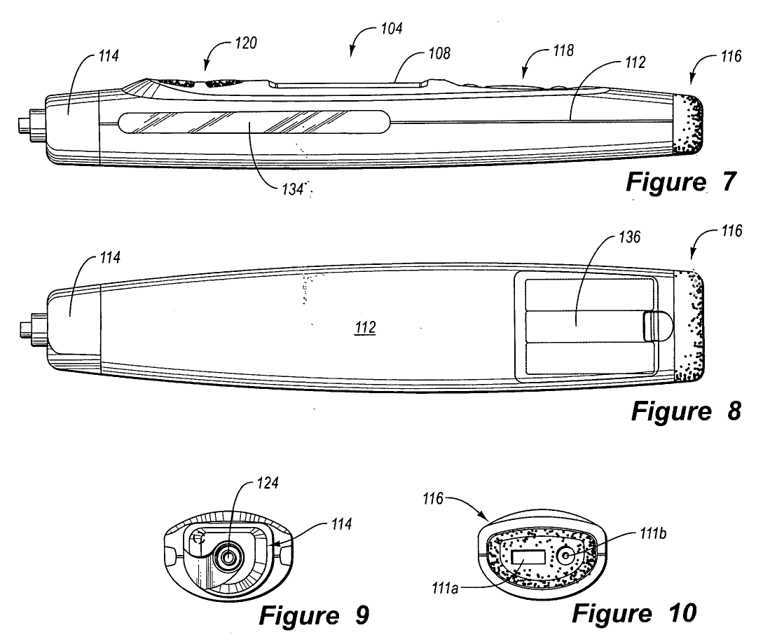

As perhaps best seen in FIG. 7, a transparent window 134 is provided along both sides of body 112 so that the user can easily see the progressive dispensing of medication within vial, cartridge, or other container 122. The bottom surface of body 112 may include a door 136 for accessing a battery compartment. FIGS. 9 and 10 illustrate the distal front and proximal rear ends of the device, respectively, showing connector 124 at distal end 114 and a data (e.g., USB) port 111a and a battery charging port 111b at proximal end 116.

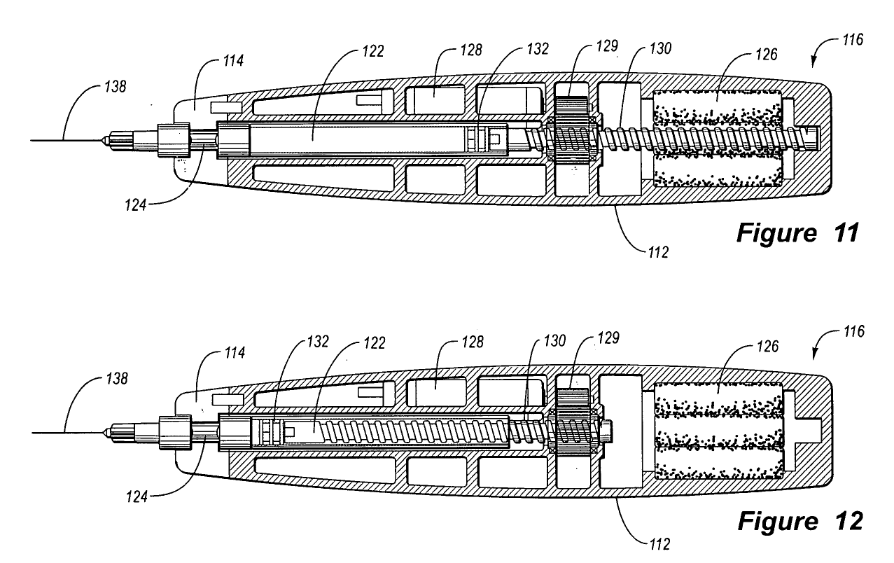

FIG. 11 and FIG. 12 illustrate cross-sections of the carrier device 104 with vial 122 fully loaded and plunger shaft 130 in a retracted position (FIG. 11) and vial 122 completely emptied and plunger shaft 130 in a fully extended position (FIG. 12).

### IV. Exemplary Method of Use

The computer 102 in the central dispensing station 100 has access to medical orders in the form of medication schedules associated with particular patients. An authorized operator accesses the computer 102, places an empty hand-held carrier 104 in the data port 106, and selects an order to apply to the carrier 104. The dispensing system responds by allowing access to the selected medication, preparing the carrier mechanically to receive the vial (e.g., unlocking carrier 104 so as to allow loading), and programming the carrier device 104 with the patient, medication, and dosage information as ordered. Such information may be displayed on display 108.

The central depository station may include a separate charging station (e.g., other than the integral charging port shown data port interface 106) which permits multiple carriers to dock in base unit receptacles that only serve only to maintain a charge on the batteries in each carrier device 104.

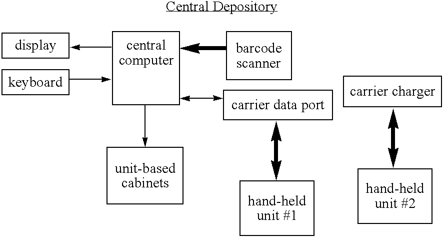

The above diagram shows the essential components of the system located at the central depository station. The diagram shows two hand-held carrier units, one being programmed and one being charged only. The central depository station may include a barcode or other ID scanner to confirm the type and dosage of the medication selected by the operator before it is loaded into the carrier (e.g., the user may scan a barcode on the container of the medication).

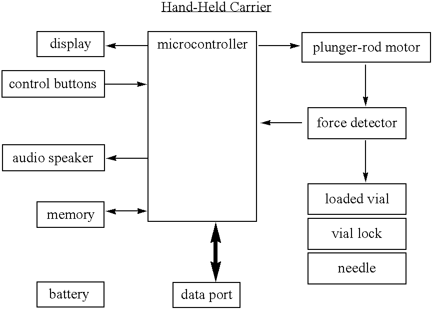

The diagram above shows the elements contained in an exemplary hand-held carrier device 104. The microcontroller interfaces with the components on the left, namely the display, control buttons, audio speaker, memory, as well as the data port. It also drives the motor and receives input from a force detector. Control buttons allow the operator to mix medications as necessary, aspirate and inject or otherwise dispense the contents of the vial, and play audio announcing the medication type and dosage programming into the unit and/or visually display this information.

Pharmacy personnel places the hand-held carrier 104 in the data port interface 106 at the central depository station 100. The computer 102 recognizes the carrier 104 and confirms that a data connection is established. The operator reviews a list of injections and/or controlled substance administrations scheduled (e.g., in the next hour) and selects a scheduled administration by earliest time, by patient, or some other criteria. Upon selection, the system confirms that inventory exists to fill the order and prepares to allow access to the proper drawer and/or bin of the cabinet 103 of station 100. The cabinets of station 100 may contain both pills and IV vials. Depending on the configuration of the hand-held carrier device received within port 106, the system may list only injection orders in an embodiment where the carrier is configured only for delivery of injection medications.

The operator activates the enabled selection, removes the vial from the cabinet 103, and scans it with a bar code wand attached to the computer 102. If the bar code does not match the expected contents of the bin, the system indicates the error; otherwise the correct vial has been accessed. The cradled hand-held carrier unit 104 responds by fully extending a plunger shaft (see shaft 130 of FIG. 11) to reveal the plunger-rod threads to mate with the plunger in the vial.

The operator breaks the seal on the accessed vial 122, attaches a luer lock needle with cover (where the contents of the vial is to be injected), and places the vial 122 in the hand-held carrier device 104 (FIG. 4). This is accomplished by rotating the distal front end 114 of the carrier 104 to allow the carrier 104 to receive the vial 122, placing the vial 122 in the carrier 104, and screwing the vial 122 onto the receiving threads of the motor-driven plunger shaft 130. At this stage the vial 122 extends well past the end of the hand-held carrier 104. The operator issues a “Load” command from the computer and/or carrier 104 (e.g., by pressing a button on carrier device 104) which causes the plunger 132 to retract, thus pulling the vial 122 into the carrier 104. A positional sensor detects when the vial 122 is positioned fully in the carrier 104 and causes the plunger shaft 130 to stop moving. At this point, the operator manually rotates the vial lock (i.e., distal front end 114) to the closed position locking the vial 122 in place so the plunger shaft 130 can operate either by retracting in or extending out as commanded later by the nurse.

Closing the vial locking mechanism with a vial confirmed as present inside the carrier sends a signal to the computer of the central depository station that the vial 122 is in place. This triggers the computer 102 to program the carrier with the injection order, including the patient, the room, the medication type and dosage. Of course, this information could alternatively be programmed into the carrier 104 prior to locking vial 122.

The nurse then takes the hand-held carrier device 104 to the patient and uses a bar code scanner in the carrier 104 to confirm that the patient matches the order programmed into the carrier (e.g., by scanning the patient's ID band). The patient's weight may also be used to confirm dosage information (e.g., typically dosage of a medication is directly linked to patient weight). If the patient scanned does not match the order, then the display may show “Error” on display 108 and provide additional visual/auditory signals (e.g., by a blinking light and/or the unit beeping repeatedly, for example five times). If the patient matches, then the carrier 104 shows the order on the display 108 and may also provide an additional signal (e.g., beeps once) to confirm. Pressing the centrally located display control button 118 causes the unit to audibly announce the medication and dosage. Pressing right or left arrow buttons 118 at this time may cause the announcement volume to temporarily increase or decrease. When the carrier is programmed at the central depository station, it may set the volume to a nominal value so the sound will always initially be enabled at a predictable level.

Once the order is confirmed and the patient matches the order, the nurse administers the injection. The nurse removes the protective cover from the needle 138. The nurse holds the needle vertically oriented upwards and presses the inject button 120 until liquid squirts out the needle 138. This pushes air out of the adapter 124 between the vial 122 and the needle 138 and purges the needle. The nurse then places the needle 138 into the patient. The nurse then presses and holds the pull-back button 120 to aspirate. The carrier 104 responds by pulling back a fixed distance on the plunger shaft 130 and holding. The nurse then inspects the distal end of the vial 122 behind the needle 138 for blood (e.g., through window 134. If there is no blood visible, then the nurse releases the pull-back button 120 and the plunger shaft 130 returns to its original position. If there is blood visible, then the needle 138 is in a blood vessel and must be relocated. During the aspiration cycle, the display 108 include a spiraling graphic (e.g., representing a “backwards” rotation of threaded plunger shaft 130) to indicate that the aspiration is in process, and then stopping at the end of the aspiration cycle. Alternatively, the user may be able to view the backwards rotational motion of the threaded plunger shaft 130 (as well as it's reverse axial movement) through window 134. Similarly, the user may view the forward rotational motion of threaded plunger shaft 130 through window 134 as the medicine is delivered to the patient. Display 108 could also be configured to provide a “forward” spiraling graphic during delivery of the medicine to the patient.

It is advantageous for the plunger stem 130 to return automatically to the starting point as this establishes the base, zero-calibration point for measuring the amount of medicine delivered. This also prevents air from entering the needle 138 if blood is detected and it becomes necessary to pull the needle out and reinsert it in a different location.

If blood is not detected during the aspiration cycle, then the nurse delivers the medication by pressing and holding the inject arrow button 120. Pressing the inject arrow button 120 (the distally located of buttons 120) may advantageously deliver the medication at a slow, steady rate. Releasing the inject arrow button 120 may pause the delivery of medication. Because there is a direct relationship between the plunger displacement and the amount of medication delivered, the carrier 104 is able to measure the amount of medication delivered, and since it was programmed with the ordered dosage, injection will automatically stop when that amount of medication has been delivered. Also during the injection, the display 108 may shows information (e.g., a dial or other indicator) indicating the force on the plunger 132. Excessive force indicates that there is resistance at the injection site which may be cause to stop the injection.

In the situation where it is desired to combine medications within the vial or other container prior to the injection, the central depository operator (e.g., pharmacist) selects a vial containing the desired amount of the primary medication. The vial is only partly full in the sense that there is room for additional secondary medicament to be aspirated into the vial and mixed with the original contents of the vial. The vial is loaded into the carrier as described previously. The needle is exposed and the seal membrane of the secondary medication vial is impaled on top of the exposed needle of the carrier. The operator issues the command to aspirate the secondary medicament. The carrier device responds by drawing in liquid from the secondary vial in the amount specified by the order in the computer. The vial in the carrier is now properly loaded and contains the desired mixture of medications. At the end of the aspiration cycle, the computer may program the carrier with the patient, medication, and dosage and indicates that the carrier is ready for use on the specified patient. Note that this mechanism allows mixing only at the central depository station.

The carrier device 104 preferably allows easy viewing of the contents of the vial 122 directly behind the needle 138 in order to determine if the needle is in a blood vessel when aspirating. Having a clear view of the entire vial 122 allows feedback to the nurse as to the position of the plunger 132 at all times during the injection. The distal end portion of the carrier may be formed of tapered thin and transparent plastic. A design of this type would allow viewing to the distal end of vial 122 and into connector 124 and possibly even the proximal end of needle 138.

Pharmacy personnel use a bar code reader to move medications into pharmacy inventory so the central computer 102 will indicate the additional inventory. In response to doctor orders, personnel in the pharmacy connects an empty carrier 104 to a computer data port interface 106 on the network, removes the appropriate medication from inventory, and places it in a carrier 104. During this process the bar code on the vial is scanned and information relating to medication type and dosage is communicated to the central database and also to the carrier memory via a wireless or wired interface (e.g., information may be relayed to carrier 104 through a wireless interface rather than the illustrated wired data port interface 106).

It will be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A system for securely dispensing and maintaining accurate inventory of controlled substances in a hospital or similar setting, comprising:
a central depository station for storing and monitoring inventory and dispensing of a plurality of medications comprising:
a computer for controlling operator access to a plurality of medications contained within the central depository station;
a data port interface for connecting a portable hand-held medication carrier device to the central depository station; and
one or more portable hand-held medication carrier devices connectable to the data port interface of the central depository station.

2. A system as recited in claim 1, wherein each portable hand-held carrier device includes an axial recess configured to receive a container of controlled substance medication.

3. A system as recited in claim 2, wherein the axial recess is cylindrical so as to be configured to receive a cylindrical vial of controlled substance medication.

4. A system as recited in claim 1, wherein each portable hand-held carrier device includes means for securely locking a container of controlled substance medication once received within the carrier device.

5. A system as recited in claim 1, wherein each portable hand-held carrier device comprises an interface including a data connector for connecting to the data port interface.

6. A system as recited in claim 5, wherein the interface of each portable hand-held carrier device further comprises a power connector adjacent to the data connector for charging a battery within the carrier device.

7. A system as recited in claim 6, wherein the interface of each portable hand-held carrier device is located at a proximal end thereof such that the portable hand-held carrier device may be received upright within the data port interface of the central depository station.

8. A system as recited in claim 6, wherein the central depository station further comprises a secondary charging interface separate from the data port interface for charging a power supply of each carrier device connected to the secondary charging interface but not connected to the data port interface of the central depository station.

9. A system for securely dispensing and maintaining accurate inventory of controlled substances in a hospital or similar setting, comprising:
a central depository station for storing and monitoring inventory and dispensing a plurality of medications comprising:
a central depository computer for providing access to a central depository management database, the computer controlling operator access to a plurality of medications stored within the central depository station;
a data port interface for connecting a portable hand-held medication carrier device such that the hand-held medication carrier device can communicate with the computer of the central depository station when the carrier device is connected to the data port interface;
one or more portable hand-held medication carrier devices connectable to the data port interface of the central depository station, each carrier device being configured to securely hold medication loaded therein at the central depository station for secure transport from the central depository station to a patient room; and
one or more patient room network terminals, each terminal being located within a patient room so as to allow a user to connect a portable hand-held medication carrier device to the central depository management database in a patient room where medication within the carrier device is to be administered.

10. A system as recited in claim 9, wherein each portable hand-held carrier device comprises an electronic display.

11. A system as recited in claim 9, wherein each portable hand-held carrier device comprises a microcontroller.

12. A system as recited in claim 9, wherein each portable hand-held carrier device comprises a threaded plunger shaft coupled to a motor for driving the threaded plunger shaft.

13. A system as recited in claim 12, wherein each portable hand-held carrier device includes a cylindrical axial recess configured to receive a cylindrical vial of controlled substance medication, a distal end of the threaded plunger shaft being configured to threadably couple to a received vial.

14. A system as recited in claim 13, wherein each portable hand-held carrier device comprises a transparent window through which the user may view substantially the full length of the vial and at least a portion of the threaded plunger shaft so as to easily view aspiration of blood at a distal end of the vial as well as rotational and/or axial motion of the threaded plunger shaft.

15. A system as recited in claim 13, further comprising a connector coupleable to a distal end of the vial and a needle coupleable to a distal end of the connector.

16. A system for securely dispensing and maintaining accurate inventory of injectable controlled substances in a hospital or similar setting, comprising:
a central depository station for storing a plurality of injectable medications comprising:
a central depository computer for providing access to a central depository management database, the computer controlling operator access to a plurality of injectable controlled substance medications stored within the central depository station;
a data port interface for connecting a portable hand-held medication carrier device such that the hand-held medication carrier device can communicate with the computer of the central depository station when the carrier device is connected to the data port interface;
one or more portable hand-held medication carrier devices connectable to the data port interface of the central depository station, each carrier device being configured to securely receive a container of injectable controlled substance medication that is locked within carrier device at the central depository station for secure transport from the central depository station to a patient room; and
one or more patient room network terminals, each terminal being located within a patient room so as to allow a user to connect a portable hand-held medication carrier device to the central depository management database in a patient room where injectable controlled substance medication within the carrier device is to be administered.

17. A method of securely dispensing and maintaining accurate inventory of controlled substances in a hospital or similar setting, comprising:
connecting an empty portable hand-held medication carrier device to a data port interface of a central depository station;
accessing a computer at the central depository station so as to select a medication order to apply to the carrier device, the central depository station allowing access to a plurality of medications stored within the central depository station so that the user may remove a selected medication for secure insertion within the hand-held medication carrier device;
loading and locking the selected medication into the hand-held medication carrier device; and
programming the carrier device with patient, medication, and dosage information relative to the selected medication order.

18. A method as recited in claim 17, further comprising:
disconnecting the loaded and locked portable hand-held medication carrier device from the data port interface of the central depository station;
carrying the hand-held carrier device to a patient that is remote from the central depository station;
confirming that the identity of the patient matches the selected medication order loaded and locked within the carrier device;
administering the medication to the patient;
connecting the carrier device to a room terminal networked to the central depository station computer so as to update a central depository management database with information relating to the completion of the medication administration; and
returning the hand-held carrier device to the central depository station.
